(19) United States
(12) Patent Application Publication
(10) Pub. No.: US 2007/0184437 A1
(43) Pub. Date: Aug. 9, 2007

(54) Method for the detection of legionella-type bacteria

(75) Inventors: Antje Breitenstein, Halle (DE); Peter Neubauer, Oulu (FI); Tarja Rosilainen, Oulu (FI)

Correspondence Address: Horst M Kasper, 13 Forest Drive, Warren, NJ, 07059, US

(73) Assignee: SCANBEC GMBH, HALLE (DE)

(21) Appl. No.: 10/554,238

(22) PCT Filed: Aug. 13, 2004
(86) PCT No.: PCT/DE04/01821

§ 371 Date: Dec. 11, 2006

(30) Foreign Application Priority Data
Aug. 15, 2003 (DE) 103 38 123.6

(51) Int. Cl. C12Q 1/68

(52) U.S. Cl. 435/6

(57) ABSTRACT

The invention concerns a method for the detection of bacteria of the genus Legionella using a sandwich hybridization procedure at temperatures between 50 and 55° C.

According to the invention new oligonucleotide probes are utilized as capture and detection probes enabling a genus or species specific detection of different Legionella bacteria.

Advantageous is the exchangeability of capture and detection probes against each other for genus or species specific detections of Legionella bacteria. Furthermore the detection procedure can be performed with combinations of oligonucleotide probes for a genus and species specific detection.

The invention concerns a method for the detection of bacteria of the genus Legionella by means of a sandwich hybridization procedure.

Legionella spp. are gram-negative, rod-shaped and facultative intracellular pathogens. Nowadays more than 42 species with 64 serogroups have been classified to belong to the genus Legionella.

They are normally found in aquatic environments or wet soil surviving as intracellular parasites of amoebae and ciliates. They are also found in man-made aquatic environments like cooling towers, clinical respiratory devices, whirlpools and showers.

People get infected with Legionella after inhaling aerosols of contaminated water droplets from the above mentioned water sources. In lungs Legionella bacteria invade to alveolar magrophages and may cause a kind of pneumonia, known as Legionnaires' disease. Symptoms are beginning mild cough, malaise, muscle aches, low fewer and gastrointestinal symptoms and later high fewer, alveolitis and bronchiolitis. Legionella (L.) pneumophila is the main causative agent of legionellosis but other species such as L. longbeachae, L. micdadei, L. dumoffii and L. bozemanii are also known to cause disease in humans.

To detect Legionella bacteria from environmental water samples the following methods are known from the literature: (i) cultivation based detection of the bacterial cells on selective media, (ii) polymerase chain reaction (PCR) based detection methods, (iii) in situ hybridization with fluorescence labelled oligonucleotide probes and (iv) monoclonal antibody based detection principles.

Furthermore the use of the sandwich hybridization method is known from U.S. Pat. No. 5,569,568 A. This method is based on the use of two oligonucleotide probes, a capture and a detection probe. The capture probe is covalently linked to a solid surface. At first the target nucleic acid hybridizes with the capture and detection probe at a specific hybridization temperature. Afterwards the target RNA—probe complex is bounded to the solid surface. The detection of the hybridization complex can be performed with fluorescent, chemiluminescent, colorimetric or radioactive signal read-out. Important for the success of this method are the properties of the respective oligonucleotide probes used for hybridization. Currently, this is only partially provided by the present state of the art techniques.

The task of this invention consists in the development and application of novel genus and species specific oligonucleotide probes for the detection of Legionella bacteria.

According to the invention this task is fulfilled as follows:
a) For the genus specific detection of Legionella a capture probe with the sequence 5′-CCTCCTCCCCACTGAAAGT-3′ and a detection probe with the sequence 5′-CACTGTATGTCAAGGGTAGG-3′;
b) for the specific detection of Legionella pneumophila a capture probe with the sequence 5′-ATCTGACCGTCCCAGGTT-3′ and a detection probe of the sequence 5′-TTCGCCGCCCTCTGTATCG-3′;
c) for the specific detection of Legionella feelei a capture probe with the sequence 5′-GCGCCACTAACCTCATTCAT-3′ and a detection probe of the sequence 5′-TATACAACCACCTACGCACC-3′;
d) for the specific detection of Legionella jordanis a capture probe with the sequence 5′-CCACTCCTCCCCACTGAAAG-3′ and a detection probe of the sequence 5′-CTTACGGTCCCCAGCTTTTT-3′
   are used and the hybridization reaction is performed at temperatures between 50 and 55° C.

The most important data of the new oligonucleotide probes are shown in the following table 1.

| | | |
|---|---|---|
| Position | Kind of | |
| in E. coli | probe in | Specific |

| | | | | |
|---|---|---|---|---|
| Name of | Target | 16S | sandwich | hybridization |

| | | | | | |
|---|---|---|---|---|---|
| the probe | Sequence | species | rDNA | hybridization | temperature |

| | | | | | |
|---|---|---|---|---|---|
| legpneu1 | 5′-TTCGCCGCCCTCTGTATCG-3′ | L. | 575-594 | detection | 50° C. |
| legpneu2 | 5′-ATCTGACCGTCCCAGGTT-3′ | pneumophila | 626-643 | capture | |
| legfeel1 | 5′-GCGCCACTAACCTCATTCAT-3′ | L. | 840-859 | capture | 55° C. |
| legfeel2 | 5′-TATACAACCACCTACGCACC-3′ | feelei | 575-594 | detection | |
| legjor1 | 5′-CTTACGGTCCCCAGCTTTTT-3′ | L. jordanis | 192-211 | detection | 55° C. |
| legjor2 | 5′-CCACTCCTCCCCACTGAAAG-3′ | | 435-454 | capture | |
| legall 11 | 5′-CCTCCTCCCCACTGAAAGT-3′ | Genus | 433-451 | capture | 50° C. |
| legall 22 | 5′-CACTGTATGTCAAGGGTAGG | Legionella | 983-1001 | detection | |

For species specific detections of Legionella species the oligonucleotides used as capture and detection probes are according to patent claim 2 exchangeable against each other.

Furthermore the genus specific detections of Legionella bacteria can be performed according to patent claim 3 with combinations of oligonucleotide probes for the genus and species specific detection, respectively.

Additionally of advantage is, that the novel genus and species specific oligonucleotide probes are developed for hybridizations at uniform temperatures between 50 and 55° C. This allows combinations of more than 2 probes, which is the premise for a detection of more than 1 Legionella species in one sample. For this paramagnetic beads coated with different oligonucleotides used as capture probes for a species specific detection of several Legionella species are mixed (multiplex analysis). For example a capture probe for the specific detection of Legionella pneumophila can be combined with capture probes for the specific detection of Legionella feeli or used in every other possible combination with a genus specific detection probe.

The invention based method is explained with reference to the enclosed picture.

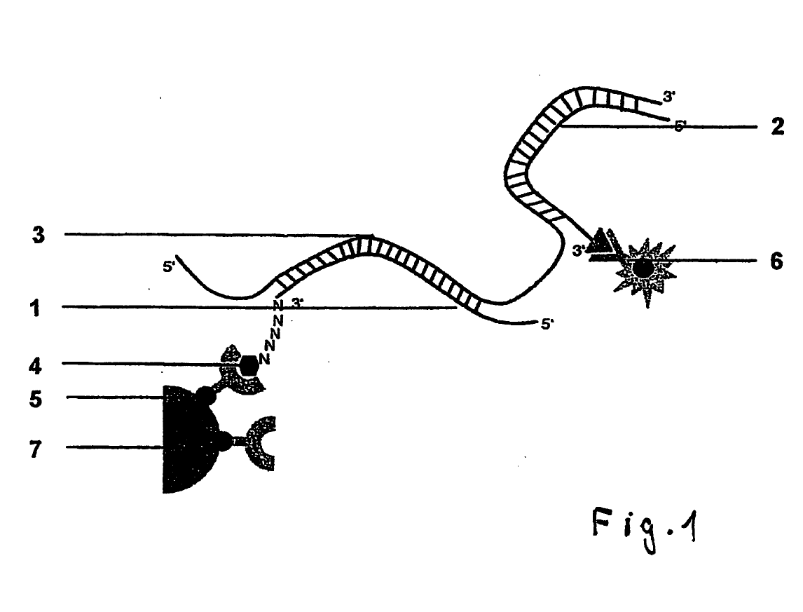

FIG. 1, page 7 is showing a schematic description of the sandwich hybridization method.

The Biotin labelled (4) capture probe (1) binds to the Streptavidin (5) coated paramagnetic beads (7). After specific hybridization of the target nucleic acid (3) with capture (1) and detection probe (2) the Alkaline Phosphatase (6) is bound to the detection probe (2) via Digoxigenin performing the fluorescence signal generation. The enzymatically amplified fluorescence signal can be quantified by means of a fluorescence reader. Another opportunity for measuring the Alkaline Phosphatase activity is the use of electrochemical sensors.

For sample preparation total DNA of different Legionella species was used. The 16S ribosomal DNA (rDNA) was amplified from total DNA of different Legionella species by means of PCR using the universal eubacterial primers fD1 and rP2.

The promotor sequence of the T7 RNA polymerase was part of the forward primer fD1. The in vitro transcribed 16S rRNA was produced from the respective PCR products (16S rDNA) of the different Legionella species using the DIG RNA Labeling Kit (SP6/T7) (Roche) or the MAXIscript Kit (Ambion), respectively.

The efficiency of the oligonucleotide probes was tested using the Slot Blot method. In vitro transcribed 16S rRNA (RNA of the small subunit of the bacterial ribosom) was used as target molecule.

The Slot Blot hybridizations were performed according to the protocol of the DIG users guide for filter hybridization (Boehringer Mannheim, 1995). 1000 fmol in vitro transcribed 16S rRNA of different Legionella species was dissolved in RNA dilution buffer (DEPC H₂O, 20×SSC, Formaldehyd (5:3:2)) and denatured for 10 min at 65° C. Afterwards the samples were applied onto a positively charged nylon membrane (Hybond N) using a vacuum Slot Blotting device. Before and after the blotting process the membrane was washed with 20×SSC (3M sodium chloride, 300 mM sodium citrate, pH 7.0). Afterwards the nucleic acids were crosslinked onto the membrane by an UV light exposure for 2 minutes.

The prehybridization reaction was performed in High SDS buffer (7% SDS; 50% formaldehyde; 5×SSC; 2% Blocking reagent (Roche); 50 mM sodium phosphate, pH 7.0; 0.1% lauroylsarcosine) for two hours at hybridization temperature. Afterwards the hybridization reaction was performed ovemight with 100 pmol DIG labelled oligonucleotide probe at 50-55° C. dependent on the melting temperature of the probe. The oligonucleotide probes were DIG labelled at the 3′ end with the DIG oligonucleotide 3′ Labelling kit (Roche) according to the manufacturers protocol.

To remove unbounded oligonucleotide probes after hybridization the membrane was washed twice with 2×SSC; 0.1% SDS for 5 minutes, followed by two washing steps with 0.1×SSC, 0.1% SDS; 0.2×SSC, 0.1% SDS or 0.5×SSC, 0.1% SDS, respectively. Afterwards the membrane was incubated in maleic acid buffer (0.1M maleic acid, 0.15M sodium chloride, pH 7.5) with 0.3% Tween 20 for 5 minutes at room temperature. To avoid unspecific binding of the Alkaline Phosphatase the membrane was incubated for 30 minutes at room temperature in maleic acid buffer with 0.1%. Blocking reagent (Roche). In a next step Anti-Digoxigenin Alkaline Phosphatase (AP) was diluted 1:20000 in maleic acid buffer with 1% Blocking reagent. Than the membrane was incubated for 30 minutes in the antibody solution. Afterwards the membrane was washed twice for 15 minutes with maleic acid buffer containing 0.3% Tween 20 and equilibrated for 5 minutes in detection buffer (100 mM Tris-HCl, pH 9.5, 100 mM sodium chloride). CDP-Star™ (Roche) was used as substrate for the Alkaline Phosphatase and was diluted 1:100 in detection buffer and applied onto the membrane surface. Afterwards the membrane was shrink-wraped in plastic foil and incubated for 10 minutes at room temperature. After that the membrane was exposed to Hyperfilm ECL (Amersham Pharmacia Biotech) for 1 hour and later on for 10 and 5 minutes to reduce the background.

The results of the Slot Blot test were as follows:
1. Probes for the detection of the genus Legionella
   The oligonucleotide probe Legall 11 hybridized with the in vitro transcribed 16S rRNAs of all investigated Legionella species (15 species, see table 2, page 8). The binding was specific for all Legionella species.
   The oligonucleotide probe Legall 22 also hybridized specifically with the in vitro transcribed 16S rRNA of all investigated Legionella species. Both probes are applicable for a genus specific Legionella detection using a sandwich hybridization procedure with Legall 11 as capture probe, Legall 22 as detection probe and a hybridization temperature of 50° C.
2. Probes for the detection of Legionella pneumophila
   The oligonucleotide probe Legpneu 1 hybridized specifically with the in vitro transcribed 16S rRNA of Legionella pneumophila serogroup 1 ATCC33152, Legionella pneumophila serogroup 6, Legionella pneumophila Philadelphia I JR32 WT and Legionella micdadei.
   The oligonucleotide probe Legpneu 2 was specifically hybridizing with in vitro transcribed 16S rRNA of Legionella pneumophila serogroup 1 ATCC33152, Legionella pneumophila serogroup 6 and Legionella pneumophila Philadelphia I JR32 WT. This probe is highly specific and therefore applicable as capture probe in combination with Legpneu 1 as detection probe within sandwich hybridization procedures performed at 50° C.
3. Probes for the detection of Legionella feelei
The probe Legfeel 1 is highly specific and was only hybridizing with the in vitro transcribed 16S rRNA of Legionella feelei. The probe can be used as capture probe in combination with Legfeel2 .for a species specific detection of Legionella feelei.
   The oligonucleotide probe Legfeel 2 is also highly specific and was only hybridizing with the in vitro transcribed 16S rRNA of Legionella feelei. The probe can be used as detection probe, due to the fact that it has a lower binding efficiency than the probe Legfeel 1.
   Both probes can be used for a species specific detection of Legionella feelei within sandwich hybridization experiments at a specific hybridization temperature of 55° C.
4. Probes for the detection of Legionella jordanis
   The oligonucleotide probe Legjor 2 hybridized with the in vitro transcribed 16S rRNA of Legionella jordanis and Legionella feelei. The binding with the Legionella jordanis sample was specific whereas the binding with the Legionella feelei sample was unspecific. This oligonucleotide probe can be used as detection probe in combination with the Legjor 1 probe.
   The oligonucleotide probe Legjor 1 only hybridized with the in vitro transcribed 16S rRNA of Legionella jordanis. The binding to the respective target molecule was specific. The probe can be used as capture probe in combination with the probe Legjor2 for sandwich hybridization experiments for the species specific detection of Legionella jordanis at a hybridization temperature of 55° C.

The advantages of the invention are that the new oligonucleotide probes are particularly suitable for the sandwich hybridization method. Additionally, it is of advantage, that one can use combinations of the novel oligonucleotide probes. Other combinations, for example with other oligonucleotides, e.g. for the useage as PCR primers, microscopic detection methods with fluorescence labelled probes, for a fluorescence sandwich hybridization procedure and as well for sandwich hybridization procedures with elecrical signal read-out, are also possible.

[image]

TABLE 2
Investigated Legionella species

| Legionella | PROBES | | | | | | | |
|---|---|---|---|---|---|---|---|---|
| species | Legall11 | Legall22 | Legpneu1 | Legpneu2 | Legfeel1 | Legfeel2 | Legjor2 | Legjor1 |
| L. bozemanii | 0 | 0 | | | | | | |
| L. dumoffii | 0 | 0 | | | | | | |
| L. erythra | 0 | 0 | | | | | | |
| L. feelei | 0 | 0 | | | 0 | 0 | 2 | |
| L. gormanii | 0 | 0 | | | | | | |
| L. hackeliae | 0 | 0 | | | | | | |
| L. israelensis | 0 | 0 | | | | | | |
| L. jordanis | 0 | 0 | | | | | 0 | 0 |
| L. longbeachae | 0 | 0 | | | | | | |
| L. micdadei | 0 | 0 | 1 | | | | | |
| L. oakridgensis | 0 | 0 | | | | | | |
| L. pneumophila | 0 | 0 | 0 | 0 | | | | |
| SG1 ATCC 33152 | | | | | | | | |
| L. pneumophila | 0 | 0 | | | | | | |
| gorby WT | | | | | | | | |
| L. pneumophila | 0 | 0 | 0 | 0 | | | | |
| Philadelphia JR32 | | | | | | | | |
| WT | | | | | | | | |
| L. pneumophila | 0 | 0 | 0 | 0 | | | | |
| Philadelphia SG6 | | | | | | | | |
| Hybridization | 50 | 50 | 50 | 50 | 55 | 55 | 55 | 55 |
| temperature | | | | | | | | |
| Washing | A | A | B | B | A | A | A | A |
| buffer | | | | | | | | |

1. Method for the detection of bacteria of the genus Legionella by means of a sandwich hybridization procedure characterized as follows:
a) For the genus specific detection of Legionella an oligonucleotide probe of the sequence 5′-CCTCCTCCCCACTGAAAGT-3′ is used as detection probe and an oligonucleotide probe of the sequence 5′-CACTGTATGTCAAGGGTAGG-3′ is used as capture probe.
b) For the species specific detection of Legionella pneumophila an oligonucleotide probe of the sequence 5′-ATCTGACCGTCCCAGGTT-3′ is used as capture probe and an oligonucleotide probe of the sequence 5′-TTCGCCGCCCTCTGTATCG-3′ is used as detection probe.
c) For the species specific detection of Legionella feelei an oligonucleotide probe of the sequence 5′-GCGCCACTAACCTCATTAT-3′ is used as capture probe and an oligonucleotide probe of the sequence 5′-TATACAACCACCTACGCACC-3′ is used as detection probe.
d) For the species specific detection of Legionella jordanis an oligonucleotide probe of the sequence 5′-CCACTCCTCCCCACTGAAAG-3′ is used as capture probe and an oligonucleotide probe of the sequence 5′-CTTACGGTCCCCAGCTTTT-3′ is used as detection probe.
All hybridizations are performed at temperatures between 50 and 55° C.

2. Method according to patent claim 1 characterized thereby, that for a species specific detection of different Legionella species the oligonucleotide probes used as the respective capture and detection probes are exchangeable against each other.

3. Method according to patent claim 1 characterized thereby, that the detection can be performed with combinations of oligonucleotide probes for a genus and a species specific detection.


SEQUENCE LISTING (8 sequences)

SEQ ID NO: 1 - DNA, 19 bp, Legionella pneumophila
ttcgccgccctctgtatcg

SEQ ID NO: 2 - DNA, 18 bp, Legionella pneumophila
atctgaccgtcccaggtt

SEQ ID NO: 3 - DNA, 20 bp, Legionella feelei
gcgccactaacctcattcat

SEQ ID NO: 4 - DNA, 20 bp, Legionella feelei
tatacaaccacctacgcacc

SEQ ID NO: 5 - DNA, 20 bp, Legionella jordanis
cttacggtccccagcttttt

SEQ ID NO: 6 - DNA, 20 bp, Legionella jordanis
ccactcctccccactgaaag

SEQ ID NO: 7 - DNA, 19 bp, Legionella sp.
cctcctccccactgaaagt

SEQ ID NO: 8 - DNA, 20 bp, Legionella sp.
cactgtatgtcaagggtagg